(19) United States
(12) Patent Application Publication
(10) Pub. No.: US 2006/0173543 A1
(43) Pub. Date: Aug. 3, 2006

(54) Support device for vertebral fusion

(76) Inventors: Salvador A. Brau, Los Angeles, CA (US); Michael L. Schiffman, Los Angeles, CA (US)

Correspondence Address: FULWIDER PATTON, 6060 CENTER DRIVE, 10TH FLOOR, LOS ANGELES, CA, 90045, US

(21) Appl. No.: 11/369,703

(22) Filed: Mar. 7, 2006

Related U.S. Application Data
(63) Continuation-in-part of application No. 10/630,198, filed on Jul. 29, 2003, now Pat. No. 7,018,414.
(60) Provisional application No. 60/399,584, filed on Jul. 30, 2002.

(51) Int. Cl. A61F 2/44

(52) U.S. Cl. 623/17.11

(57) ABSTRACT

A support for vertebral fusion prevents subsidence and eliminates the need for posterior surgery and instrumentation. The support is constructed of an implantable man-made material. One embodiment is generally a U-shaped metal support that rests on the apophyseal ring of a patient's vertebrae, with the open portion of the U facing the patient's posterior. The metal support is connected to a previously placed threaded cage or bone dowel such as that used in anterior lumbar interbody fusion (ALIF). The U-shaped support is preferably comprised of a trabecular metal such as tantalum.

### CROSS-REFERENCE TO RELATED APPLICATION

This application claims the benefit of and is a continuation-in-part of application Ser. No. 10/630,198 filed Jul. 2, 2003, which issued as U.S. Pat. No. ______ on _{—}, 2006, and which was a continuation-in-part of Provisional Application No. 60/399,584, filed Jul. 30, 2002.

### BACKGROUND OF THE INVENTION

This invention relates to the maintenance of an adequate disc height by the prevention of subsidence following Anterior Lumbar Interbody Fusion (ALIF). Typically ALIF has often used threaded metal cages or threaded bone dowels as the sole supports. Cages and bone dowels have been used for well over a decade. Initially, these supports were expected to act as a stand-alone device that would promote fusion and maintain disc height without the need for posterior surgery and additional instrumentation of the spine. Despite fusion rates better than 90 percent for single level fusion and 65 percent for two-level fusion, significant subsidence has been observed on X-rays taken at varying times following the procedure. This subsidence, the slow insinuation of the threaded devices into the vertebral bodies, has resulted in lost disc height, which in some patients has resulted in the failure to fuse and the recurrence of often very painful symptoms.

Subsidence occurs because the threaded devices are usually placed more posterior than anterior, where the maximum load is exerted on the vertebral body. The threaded devices also need to be placed at opposite sides from the midline of the vertebral body. This typically results in the threaded devices being placed entirely on softer bone, which is more prone to result in subsidence. The apophyseal ring, a structure within the vertebral body, provides an area of denser, stronger bone that is more resistant to subsidence. This ring, however, is found only in the very outer circumference of the vertebral body. Furthermore, the apophyseal ring is present only on the anterior and lateral aspects of the vertebral body, not on the posterior aspects, and it is typically only a millimeter or two thick. Threaded devices in use today are placed inside of the apophyseal ring and fail to take advantage of its strength. ALIF and threaded cages, therefore, have been used less frequently in recent years. This has resulted in the increase of anterior-posterior fusions, or 360 degree fusions, which have actually become the “gold standard” against which other technologies are being measured for reliability and successful outcomes.

The principal disadvantage of 360 degree fusions is that the patient then needs two separate operations, either on the same day or in two separate stages. Both operations are of significant magnitude with independent, significant morbidities. There are other problems as well. Although 360 degree fusions offer almost 100 percent fusion rates, there is not 100 percent satisfaction on the part of the patients. The most frequent and important cause of patient dissatisfaction occurs because the posterior portion of the operation causes significant destabilization of the back muscles, which are essential for improved health of the patient's back. The anterior approach, especially when used with mini-open techniques, would be preferable to 360 degree fusion, because the morbidity associated with it is much less. If 360 degree fusion could be avoided, there would be significant benefit to the patient in terms of reduced morbidity and faster recuperation. This, in turn, would result in earlier resumption of physical activity and return to work. In addition, there would substantial reduction in cost of treatment, since one operation would take the place of two.

No devices or surgical methods in use at the present time can overcome the various problems associated with either traditional ALIF or 360 degree fusion. One device, described in U.S. Pat. No. 6,210,442, tries to overcome some of these problems. The device consists of a single threaded implant incorporated into a winged structure that provides lateral support. This device, however, is not designed to take advantage of the apophyseal ring and thus fails to use the stronger bone as a source of stability and strength. It would be desirable if the benefits of a single ALIF surgery could be obtained while preventing the post-operative subsidence that typically occurs afterward.

In the inventors' parent application, now issued U.S. Pat. No. ______, an allograft of cadaverous bone was used as a spacer on the anterior portion of a patient's vertebrae. While the allograft improved on traditional ALIF, they recognized that procurement of the allograft in substantial quantities could be difficult. Therefore, they sought a man-made material that could be manufactured in different sizes and still take advantage of the apophyseal ring for ALIF.

### SUMMARY OF THE INVENTION

The present invention solves the problems encountered in ALIF and avoids the problems of 360 degree fusions, because it requires only one operation and uses an anterior approach. The invention acts as a spacer placed around the threaded devices used in ALIF. Once inserted, the invention takes advantage of the strength of the apophyseal ring of the fused vertebrae. After allowing 0.5 to 1.0 mm of subsidence on each vertebral end plate, the device then shares the load at the strongest part of the vertebral body, the apophyseal ring. Further subsidence is prevented and the successful fusion rate is increased. This makes the anterior-only approach, or ALIF, acceptable as a stand-alone construct for one or two levels of vertebral fusion, thus eliminating the need for a 360 degree fusion with separate anterior and posterior operations.

In one preferred embodiment of the invention, a spinal fusion support for preventing subsidence in ALIF comprises a portion of material adapted to be placed on the apophyseal ring of a vertebrae to be fused. Preferably the material is Implex® Trabecular Metal Material, which is made from tantalum. The spinal fusion support has an anterior cross member with two lateral ends. The cross-member is generally configured in the shape of the anterior portion of the adjacent vertebral body and configured to be substantially coextensive with the adjacent vertebral body. The cross member also has a portion generally coextensive with the apophyseal ring of the adjacent vertebral body. Each of two lateral members has an anterior end and a posterior end, the anterior end being connected to one of the lateral ends of the anterior cross member. Each lateral member is generally configured in the shape of the lateral portion of the adjacent vertebral body and substantially coextensive with the lateral portion of the adjacent vertebral body, including the apophyseal ring. The posterior ends of the lateral members define a posterior opening of the support. The invention also includes a connector adapted to connect the spinal fusion support to an associated support located posterior of the anterior cross member.

Preferably the cross member and two lateral members form a unitary structure. The lateral members are attached to the cross member and define an interior and exterior of the support device. An associated support device, such as threaded cages or bone dowels used in ALIF, is placed at least partially interior of the spinal support of the present invention. The spinal support and the associated support can be made of bone or of man-made material with a good safety record for implantation, such as titanium, titanium cobalt-chromium, stainless steel, plastic, or composites.

Another embodiment of the present invention is an improved method of ALIF which includes placing a second support on the anterior and lateral aspects of the apophyseal ring of a vertebral body after a first metal support has already been placed on the vertebral body during the initial part of the ALIF. The second Support is the fusion support device, preferably comprised of trabecular metal.

Other features and advantages of the invention will become apparent from the following detailed description, taken in conjunction with the accompanying drawings, which illustrate, by way of example, the features of the invention.

### BRIEF DESCRIPTION OF THE DRAWINGS

FIG. 1 is a perspective view of a typical prior art cage mounted on a vertebral body.

FIG. 2 is a side view of a vertebral body with a typical prior art cage mounted on the vertebral body.

FIG. 3 is a plan view of a vertebral body with a typical prior art cage mounted on the vertebral body.

FIG. 4 is a perspective view of the fusion support device.

FIG. 5 is a plan view of the fusion support device.

FIG. 6 is a plan view of the fusion support device mounted on the anterior end of a vertebral body.

FIG. 7 is a perspective view of the fusion support device and cages on a vertebral body.

FIG. 8 is a side view of a prior art cage and a fusion support device placed at the anterior end of the vertebral body.

FIG. 9 is a perspective view of another embodiment of the fusion support device and cages on a vertebral body.

### DETAILED DESCRIPTION OF THE PREFERRED EMBODIMENTS

The present invention is a novel device and method designed to provide supplemental vertebral body end plate support for devices placed in the anterior lumbar region, such as threaded interbody fusion cages or bone dowels. The present invention generally contemplates two embodiments. One, an allograft, is made from an actual vertebral body. This embodiment is described and claimed in U.S. Pat. No. ______, issued _{—}, 2006, which is the parent of this application. The entire patent is incorporated herein by reference. The second embodiment is fabricated from man-made materials such as metal, plastic, or composites. The preferred material for the second embodiment is Implex® Trabecular Metal Material made from tantalum, a product of Zimmer Spine of Minneapolis, Minn. This application includes a device and method using the man-made materials and a method using the allograft of the inventors' parent application.

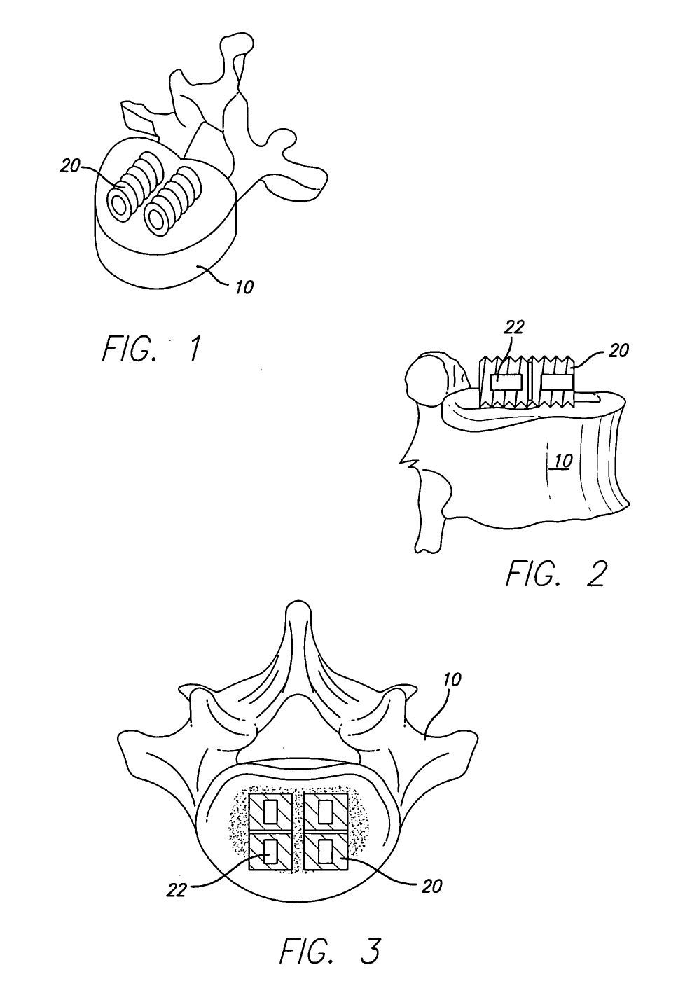

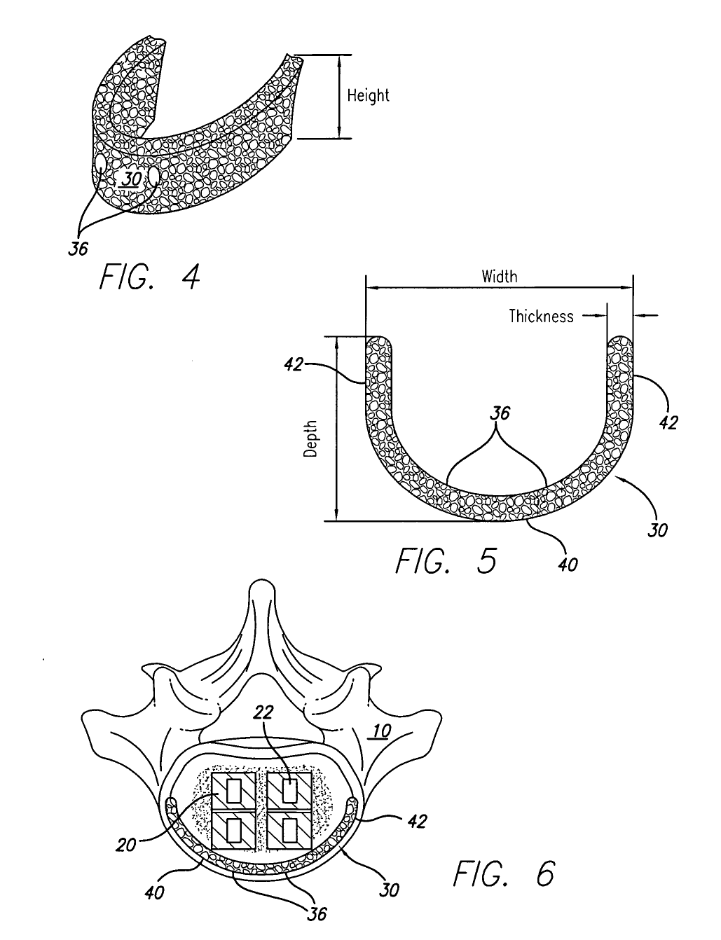

Preliminarily, the surgeon will effectively perform an ALIF, typically installing a first support device such as a pair of threaded cages or bone dowels. FIGS. 1, 2, and 3 depict different views of a vertebral body 10 with prior art cages 20 mounted on the vertebral body 10. Typically such cages contain open spaces 22 that permit bone growth throughout cage 20 for a stronger and more stable fusion. After the surgeon installs the cages, dowels, or other such support 20, he will then install a second support. In the present embodiment of the invention, the second support will be a man-made metal such as the Implex® Trabecular Metal Material. The shape of the second or spinal fusion support 30, such as that depicted in FIGS. 4-6, will conform as much as practicable to the shape of the apophyseal ring of the adjacent vertebrae, which is generally semicircular or U-shaped. The closed portion of the semicircle or U is generally co-linear and co-planar to the anterior part of the adjacent vertebral body, where the apophyseal ring is the thickest. As depicted in FIG. 5, the spinal fusion support 30 will have an anterior cross member 40 and two lateral members 42.

The second support, or spinal fusion support, 30 is preferably 1 to 3 millimeters thick. Larger thicknesses can be used, but the surgeon may have difficulty in placing them as easily as he does the thinner supports. The type of material can also affect the thickness of fusion support 30. A solid material such as titanium or steel can be manufactured to a thinner dimension. Because of the open, porous nature of Implex® trabecular material, the minimum practical thickness currently appears to be approximately 3 millimeters. One should not, however, consider the present invention so limited. Future metallurgical and manufacturing developments may permit smaller and non-uniform dimensions.

FIGS. 4 and 5 show the dimension designations of the invention. For ease of handling and manufacture, the preferred configuration of the fusion support device 30 has a constant height. To date, devices 11, 13, and 15 millimeters high have been manufactured. Again, however, the invention should not be limited to those dimensions or to a constant height, although they represent the most typical configurations that will be required. Support devices with a variety of heights, widths, and lengths can be manufactured, so that they can fit patients with different size vertebrae and intervertebral spaces. Present widths that have been manufactured range from 38 to 44 millimeters, while so far the depth has remained constant at 27.6 millimeters.

The support device can be fabricated in different sizes to match the size of the recipient's vertebrae and to match the space available once the threaded cages 20 or such similar supports are inserted into the intervertebral space. As depicted in FIG. 4, the height of the spinal fusion support, or second support 30, typically will vary to accommodate the patient. It should preferably be about 1 or 2 millimeters less than the distraction created on the vertebral end plates by the insertion of the cages, dowels, or other similar devices. This will allow a clearance of approximately 0.5-1 mm from each end plate to facilitate placement.

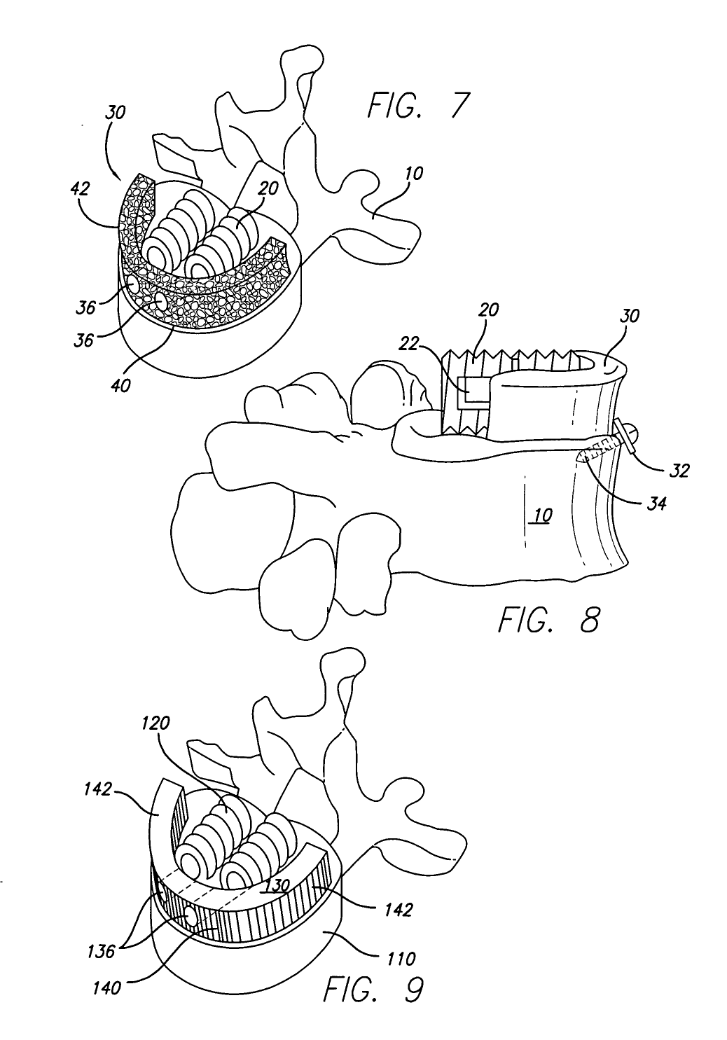

As depicted in FIG. 6, the spinal fusion support 30 will be seated at the lateral and anterior apophyseal lines of the patient's recipient vertebrae 10. This will prevent subsidence by sharing the load with fusion devices like cages and dowels, i.e., first support 20, with allowance for approximately 0.5 to 1.0 mm settling of these devices into the end plates. The generally U-shaped or semicircular spinal fusion support 30 will be inserted around the already-implanted fusion devices or first support 20. Preferably the support 30 will be secured through apertures 36 drilled through support 30. Screws, dowels, or other securing devices (not shown) will pass through apertures or holes 36 to secure the first support 20 to the spinal fusion support 30. The preferred arrangement is depicted in FIGS. 6 and 7. FIG. 8 depicts an alternative method of attachment, which avoids the necessity to drill holes 36 to attach the support body to the cages 20.

FIG. 8 depicts the vertebral allograft of the spinal fusion support 30 disclosed and claimed in the parent application. The method claimed herein can employ such a spinal fusion support 30, or a man-made material. FIG. 9 represents an embodiment similar to the trabecular or allograft spinal fusion supports 30, except the fusion support is not made of a trabecular material. The spinal fusion support 130 sits on vertebrae 110. Apertures 136 through fusion support 130 lead to cages 120, to which to which support 130 is preferably secured.

Alternatively, the screw holes in the cages, dowels, or other devices can be used to attach a bracket (not shown) that can then be used to hold the fusion support device in place and prevent it from dislodging. This embodiment was depicted in the parent application. Those of skill in the art will recognize that the attachment of the spinal fusion support 30 to a patient's vertebral body is a matter of choice and can vary, depending on the material used for the spinal fusion support, the surgeons' technique, and the physiological conditions the surgeon encounters.

It will be noted by those of skill in the art that changes may be made to the present invention without departing from its spirit or from the scope of the claims. For example, as noted above, the device can be made of a various materials. One such preferred material is titanium cobalt-chromium, although any implantable material with adequate strength could be used. The device may also be coated with material that will foster boney ingrowth for more mechanical strength and stability. Others may develop first supports that are different from the traditional threaded cages and bone dowels used in ALIF. Those of skill in the art will also understand that the dimensions provided here are only approximate, and are subject to variation depending upon the available allografts, manufacturing techniques and limitations, as well as limitations created by the patient's physique and the operating technique used by the surgeon. Likewise, the description of the invention as U-shaped or semicircular is quite general and should be interpreted broadly. For example, FIG. 5 depicts more of a U-shape. The invention, however, could be more semicircular as shown in FIG. 6, or it could be almost circular, with perhaps 270 degrees of edge and a 90 degree opening at the posterior end. Thus, the lateral members may not in a literal sense be parallel to the patient's midline, nor will the cross member be absolutely perpendicular to the midline. As a practical matter, however, they can be considered to generally posses those mathematical properties. For example, the structure might have a fairly short cross member with lateral vertebral supports that are not exactly parallel to the patient's midline. Nevertheless, for purposes of the present invention, one should still consider the structure as U-shaped, in the sense that it is supported by significant portions of the anterior and lateral aspects of the apophyseal ring. In addition, how the cross member and lateral members are attached—whether they are unitary structures or welded or slidably connected—is also a matter of choice. As imaging and fabrication techniques improve, it may, for example, be possible to use fairly rigid unitary plastic devices to conform to the precise shape of the patient's vertebrae. The same may be true of metal fusion supports. Obviously, it is preferable that the device be supported by as much of the apophyseal ring as possible. As a practical matter, however, it is necessary that the device only be supported to the extent necessary to permanently limit or prevent subsidence. Thus, the broad scope of the invention should be understood in the context of the specification and as it is defined in the following claims.

What is claimed is:

1. A spinal fusion support for placement adjacent to a vertebral body having an anterior portion, two lateral portions, a posterior portion, and an apophyseal ring, comprising:
an anterior cross member with two lateral ends,
the cross-member generally configured in the shape of the anterior portion of the adjacent vertebral body and configured to be substantially coextensive with the adjacent vertebral body, and
the cross member comprised of a man-made material having a portion generally coextensive with the apophyseal ring of the adjacent vertebral body;
two lateral members of man-made material, each lateral member having an anterior end and a posterior end, the anterior end being connected to one of the lateral ends of the anterior cross member, and
each lateral member being generally configured in the shape of the lateral portion of the adjacent vertebral body and substantially coextensive with the lateral portion of the adjacent vertebral body, and,
each lateral member being comprised of trabecular material having a portion generally coextensive with the apophyseal ring of the adjacent vertebral body, wherein the posterior ends of the lateral members define a posterior opening of the support; and
a connector adapted to connect the spinal fusion support to at least one of an associated support located posterior of the anterior cross member and the vertebral body.

2. The spinal fusion support of claim 1, wherein the associated support is adapted to be configured in different lengths.

3. The spinal fusion support of claim 2, further comprising a connector adapted to connect the lateral members to an associated support located posterior of the cross member and interior of the lateral members.

4. The spinal fusion support of claim 2, wherein the man-made material has a generally trabecular configuration.

5. The spinal fusion support of claim 4, wherein the material is comprised of tantalum.

6. The spinal fusion support of claim 4, wherein the support has a thickness of 1 to 3 millimeters.

7. The spinal fusion support of claim 5, wherein the support has a thickness of 2-4 millimeters.

8. The spinal fusion allograft of claim 6, wherein the support has a width of 38 to 44 millimeters.

9. A method of anterior lumbar interbody fusion (ALIF), comprising the steps of:
determining the width and depth dimensions of a patient's inferior and superior vertebral bodies associated with the ALIF and the dimension of the disc space between the inferior and superior vertebral bodies;
selecting a spinal fusion support of a width, depth, and height compatible with the dimensions and spacing of the inferior and superior vertebral bodies to be fused, the spinal fusion support having an anterior cross member with two lateral ends and two lateral members,
the cross-member generally configured in the shape of the anterior portion of the associated vertebral bodies and configured to be substantially coextensive with the associated vertebral bodies, and the cross member having a portion generally coextensive with the apophyseal ring of the associated vertebral bodies,
each lateral member having an anterior end and a posterior end, the anterior end being connected to one of the lateral ends of the anterior cross member, and each lateral member being generally configured in the shape of the lateral portion of the associated vertebral bodies and substantially coextensive with the lateral portion of the associated vertebral bodies, and, each lateral member having a portion generally coextensive with the apophyseal rings of the associated vertebral bodies, wherein the posterior ends of the lateral members define a posterior opening of the support;
surgically providing anterior access to the associated vertebral bodies;
surgically installing a first fusion support device; and
securing the spinal fusion support to at least one of the first fusion support device and the associated vertebral bodies.

10. The method of claim 9, wherein the first fusion support is one of cages and threaded dowels.

11. The method of claim 10, wherein the spinal fusion support is an allograft of cadaverous bone.

12. The method of claim 10, wherein the spinal fusion device is one of a group of materials including tantalum, titanium, titanium cobalt-chromium, stainless steel, plastic, and composites.

13. The method of claim 12, wherein the material has a generally trabecular configuration and is comprised of tantalum.

14. An improvement to the method of anterior lumbar interbody fusion (ALIF), in which a first support such as cages or dowels is placed on a vertebral body generally inside the anterior and lateral aspects of the apophyseal ring of the vertebral body, the improvement comprising the steps of:
placing a second fusion support on the vertebral body so that the second fusion support rests principally on a substantial portion of the anterior and lateral aspects of the apophyseal ring of the vertebral body, the second fusion support configured to be substantially coextensive with the vertebral body and having a portion generally coextensive with the apophyseal ring of the vertebral body; and
securing the second support to at least one of the first support and the vertebral body.

15. The improved method of claim 14, wherein the second fusion support comprises a unitary cadaverous vertebral allograft.

16. The improved method of claim 14, wherein the second fusion support is comprised of a man-made material with a generally trabecular configuration.

17. The improved method of claim 16, wherein the material is tantalum.
